(19) United States
(12) Patent Application Publication
(10) Pub. No.: US 2025/0235314 A1
(43) Pub. Date: Jul. 24, 2025

(54) TRANSLUMINAL DELIVERY DEVICES AND RELATED KITS AND METHODS

(71) Applicant: Merit Medical Systems, Inc., South Jordan, UT (US)

(72) Inventors: Zeke Eller, Plano, TX (US); Evi Shiakolas, Dallas, TX (US)

(21) Appl. No.: 19/034,346

(22) Filed: Jan. 22, 2025

Related U.S. Application Data
(60) Provisional application No. 63/624,002, filed on Jan. 23, 2024.
(60) Provisional application No. 63/624,084, filed on Jan. 23, 2024.

(51) Int. Cl. A61F 2/24

(52) U.S. Cl. CPC A61F 2/2436

(57) ABSTRACT

Prosthesis deployment devices and methods of a deploying a prosthesis are disclosed herein. In some embodiments, a method of deploying a prosthesis includes advancing a tip of a delivery catheter assembly positioned at a distal region of the delivery catheter assembly into one or more target structures, aligning an alignment indicator in the distal region of the delivery catheter assembly with a proximal surface of a proximal target structure of the one or more target structures, after the alignment indicator is aligned with the proximal surface of the proximal target structure actuating an actuator of a housing assembly operably connected to the delivery catheter assembly and thereby deploying a distal end of the prosthesis in the patient, and actuating the actuator of the housing assembly and thereby deploying a proximal end of the prosthesis in the patient.

### CROSS-REFERENCE TO RELATED APPLICATIONS

This application claims priority to U.S. Provisional Patent Application No. 63/624,002, entitled “TRANSLUMINAL DELIVERY DEVICES AND RELATED KITS AND METHODS,” filed on Jan. 23, 2024, and U.S. Provisional Patent Application No. 63/624,084, entitled “TRANSLUMINAL DELIVERY DEVICES AND RELATED KITS AND METHODS,” filed on Jan. 23, 2024, each of which is incorporated herein by reference in its entirety.

### TECHNICAL FIELD

This application generally relates to medical devices. More particularly, this application relates to transluminal delivery devices and related kits and methods.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates an exemplary embodiment of a prosthesis delivery device.

FIG. 2A illustrates a close-up perspective view of the prosthesis delivery device of FIG. 1.

FIG. 2B illustrates an enlarged view of the area 111b of FIG. 1.

FIG. 2C illustrates an electrode tip of the prosthesis deliver device of FIG. 1.

FIG. 2D illustrates a distal region of another embodiment of a prosthesis delivery device.

FIG. 3A illustrates a breakaway view of the thumbscrew from a distal view.

FIG. 3B illustrates a breakaway view of the thumbscrew from a proximal view.

FIG. 3C illustrates a breakaway cross-sectional view of the thumbscrew and a bushing.

FIG. 3D illustrates a cross-sectional view of the bushing of FIGS. 3A-3C.

FIG. 4A illustrates an exploded view of a housing assembly of FIG. 2A.

FIG. 4B illustrates a perspective view of the safety buttons of FIG. 2A.

FIG. 4C illustrates a breakaway view of the safety button of FIG. 2A.

FIG. 5A illustrates insertion of one embodiment of a sheath through a stomach wall and into a cyst.

FIG. 5B illustrates proximal repositioning of the sheath of FIG. 5A after insertion and before deployment of an exemplary prosthesis.

FIG. 5C illustrates partial retraction of an outer sheath of the sheath and the resulting partial deployment of an exemplary prosthesis in the cyst.

FIG. 5D illustrates one embodiment of full retraction of the outer sheath of FIG. 5C and the resulting full deployment of the exemplary prosthesis in the cyst and a stomach.

FIG. 5E illustrates insertion of one embodiment of a sheath through a stomach wall and into a cyst.

FIG. 5F illustrates proximal repositioning of the sheath of FIG. 5E after insertion and before deployment of an exemplary prosthesis.

FIG. 5G illustrates partial retraction of an outer sheath of the sheath and the resulting partial deployment of an exemplary prosthesis in the cyst.

FIG. 5H illustrates one embodiment of full retraction of the outer sheath of FIG. 5G and the resulting full deployment of the exemplary prosthesis in the cyst and a stomach.

FIG. 6 is a flow diagram of a method of deploying a prosthesis in a patient.

### DETAILED DESCRIPTION

Prosthesis deployment devices and related methods of deploying a prosthesis are disclosed herein. In some embodiments, the prosthesis deployment device comprises an elongate delivery catheter assembly configured for electrosurgery and also configured to retain and deploy a prosthesis. The prosthesis deployment device may further comprise a housing assembly operably coupled to the delivery catheter assembly and configured to connect to an electrosurgical power generator. The housing assembly may comprise an actuator configured to displace a portion of the delivery catheter assembly to deploy the prosthesis, upon actuation. The actuator may be configured for one-handed operation.

The delivery catheter assembly may include an outer sheath having an alignment indicator spaced from a distal sheath end of the outer sheath from which the prosthesis deploys. For example, the alignment indicator may be a distal end of a marker that extends proximally from the alignment indicator. In some embodiments, the alignment indicator may be a proximal end of a marker that extends distally from the alignment indicator to the distal sheath end. During use of the prosthesis deployment device, an electrode tip secured to the outer sheath of the delivery catheter assembly may by advanced through a proximal target structure (such as a stomach wall) and a distal target structure (such as a cyst wall). After the electrode tip has been advanced through the proximal target structure and the distal target structure, the alignment indicator may be aligned with a proximal surface of the proximal target structure. For example, a user may proximally pull a handle or housing assembly of the prosthesis deployment device to pull the alignment indicator back and align the alignment indicator with the proximal surface of the proximal target structure. Because the alignment indicator is spaced from the distal sheath end, when the alignment indicator is aligned with the proximal surface of the proximal target structure, the distal sheath end (and the electrode tip) are positioned at least proximate to a distal surface of the distal target structure (e.g., the distal sheath end is positioned distal to the distal target structure). This positioning of the alignment indicator with the proximal surface of the proximal target structure indicates the delivery catheter system is in the desired positioned to begin deployment of the prosthesis, and the delivery catheter may then be locked into this position with the echoendoscope.

With the distal sheath end (and the electrode tip) positioned distal to the distal target structure, actuating an actuator on the housing assembly that moves the outer sheath proximally relative to the electrode tip deploys a distal prosthesis end of the prosthesis in the patient at least proximate (e.g. adjacent) to a distal surface of the distal target structure. The actuator may be further actuated to further move the outer sheath relative to the stationary actuator tip, thereby deploying a proximal prosthesis end of the prosthesis at least proximate (e.g., adjacent) to the proximal surface of the proximal target structure.

This configuration of the delivery catheter assembly that includes the alignment indicator spaced from the electrode tip and the distal end of the outer sheath eliminates the need for a user to pull on the delivery catheter assembly once the distal prosthesis end has been deployed and is engaging with the distal target structure.

In some embodiments, the prosthesis deployment device includes only a single safety button or tab (e.g., besides the single safety button, additional safety buttons or tabs are absent from prosthesis deployment device). Conventional prosthesis deployment devices typically include two, three, four, or more safety buttons or tabs. By including only a single safety button or tab, the prosthesis deployment device according to some embodiments is easier to use. In many embodiments, the single safety button or tab is configured to selectively lock and unlock the actuator.

In some embodiments, the prosthesis deployment device includes an electrode tip including a housing comprising a distal taper and a blunt leading edge comprising a single ring electrode positioned at the blunt leading edge. The single ring electrode being the only electrode present on the tip electrode. Conventional delivery catheter assemblies typically include an electrode tip having fins or other regions that include the electrode of the electrode tip.

Kits comprising the prosthesis deployment devices with a prosthesis loaded into a prosthesis pod of the device are disclosed herein as well as methods of using the prosthesis deployment devices.

It will be readily understood that the components of the embodiments as generally described and illustrated in the figures herein could be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrases “communication with,” “engaged with,” “connected to,” and “coupled to” are used in their ordinary sense, and are broad enough to refer to any suitable coupling or other form of interaction between two or more entities, including mechanical, electrical, magnetic, electromagnetic, fluid, and thermal interaction. Two components may interact with each other even though they are not in direct contact with each other. For example, two components may be coupled to each other through an intermediate component. The directional terms “proximal” and “distal” are used herein to refer to opposite locations on a component or device. The proximal end of a component or device is defined as the end of the device closest to the practitioner when the device is in normal use by the practitioner. The distal end is the end opposite the proximal end, along the longitudinal direction of the device, or the end farthest from the practitioner during normal use.

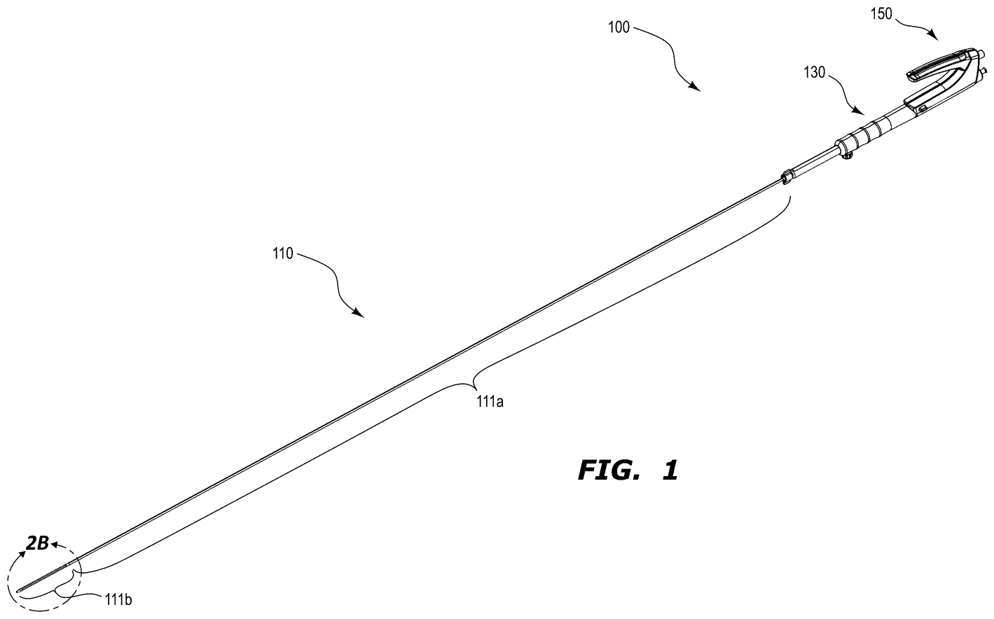

Turning now to the figures, FIG. 1 illustrates an exemplary embodiment of a prosthesis delivery device 100. The prosthesis delivery device 100 includes an elongate delivery catheter assembly configured for electrosurgery and also configured to retain and deploy a prosthesis. The prosthesis delivery device 100 includes a housing assembly 130 operably coupled to the delivery catheter assembly 110 and configured to connect to an electrosurgical power generator (not shown). The housing assembly 130 includes a handle assembly 150 configured to displace a portion of the delivery catheter assembly 110 to deploy the prosthesis 200, upon actuation of the handle assembly 150. In the illustrated embodiment, the handle assembly 150 is configured for one-handed operation.

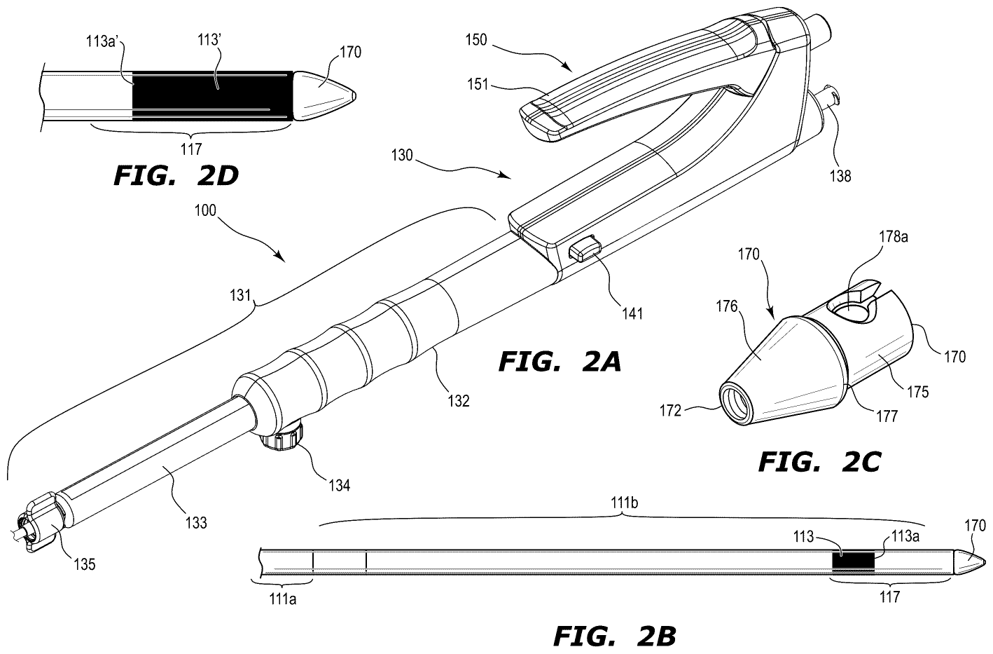

FIG. 2A illustrates a close-up perspective view of the housing assembly 130 of the prosthesis delivery device 100. The housing 130 may be operably coupled to the delivery catheter assembly and configured to connect to an electrosurgical power generator (not shown). The housing assembly 130 may include a handle assembly 150 configured to displace a portion of the delivery catheter assembly to deploy the prosthesis upon actuation of the handle assembly 150. The prosthesis delivery device 100 may include a slide assembly 131, a slide handle 132, a piston 133, a thumbscrew 134, and a female luer lock adapter 138 that may function similar to corresponding components described previously. The prosthesis delivery device 100 may further include a safety button (e.g., safety tab) 141. Other embodiments of housing assemblies and handle assemblies that may be utilized with the outer sheath described herein are disclosed in U.S. Pat. No. 11,628,078 filed on Mar. 14, 2018, the disclosure of which is incorporated herein, in its entirety, by this reference.

In the illustrated embodiment, the delivery catheter assembly 110 is configured for insertion in a working channel of an echoendoscope (not shown). Upon engagement with the echoendoscope (such as via rotatable male luer lock adapter 135), the slide assembly 131 is configured to allow adjustment of the position of the delivery catheter assembly 110 relative to the echoendoscope. For example, the tip electrode 170 may only extend slightly past the echoendoscope (referring to termination of the working channel) when the delivery catheter assembly 110 is fully inserted through the working channel. The slide assembly 131 may allow sufficient movement of the tip electrode 170 (discussed in more detail below) to perform electrosurgery and correct placement of the delivery catheter assembly 110 for deployment of the prosthesis 200. The length of the slide assembly 131, in particular the length of the piston 133, can be selected to provide the necessary travel for the tip electrode 170 beyond the end of the echoendoscope.

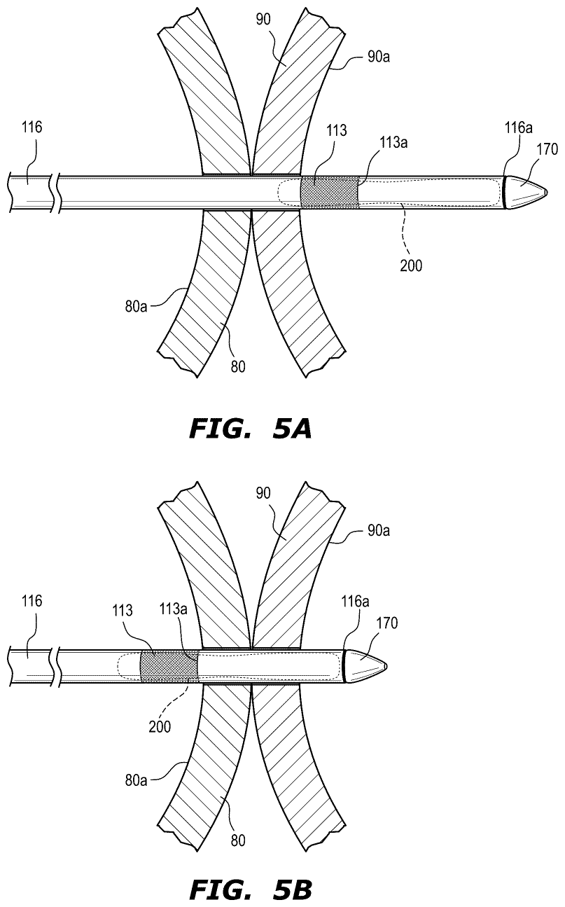

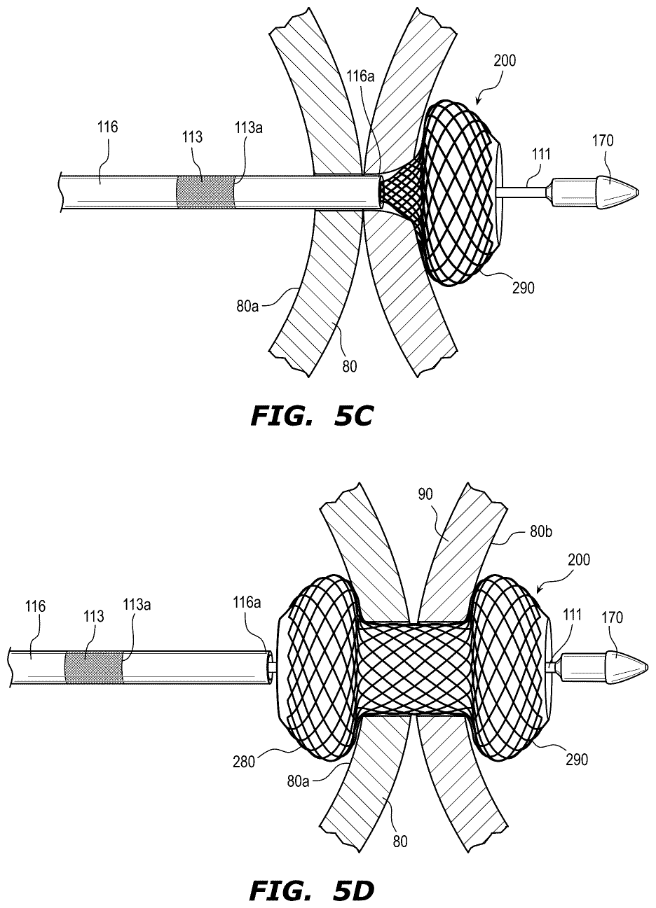

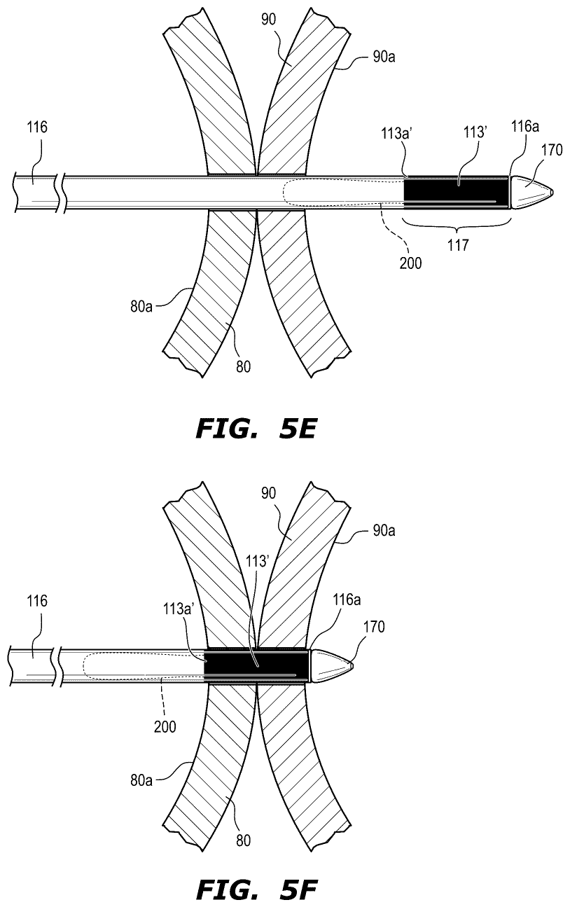

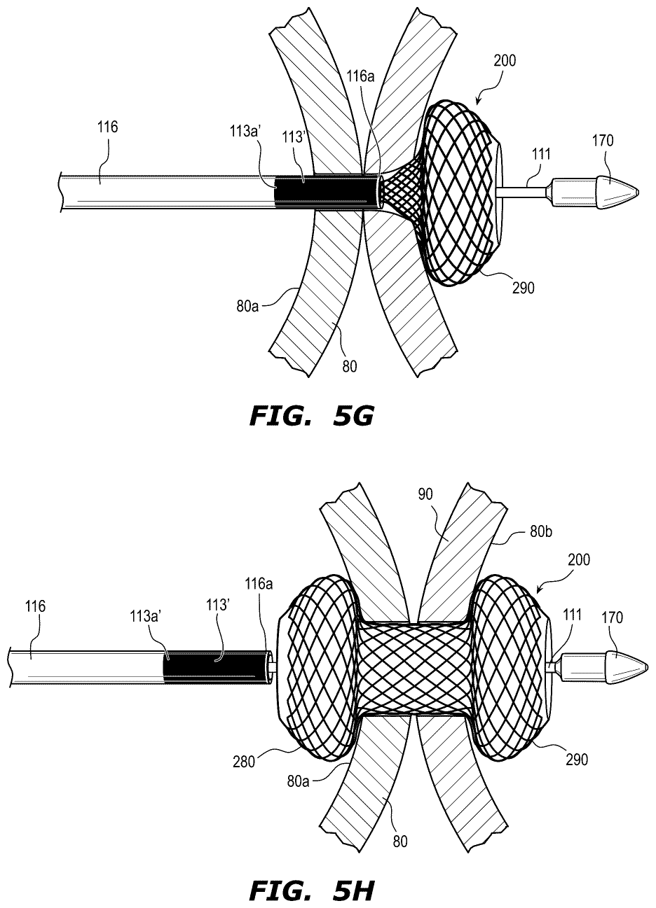

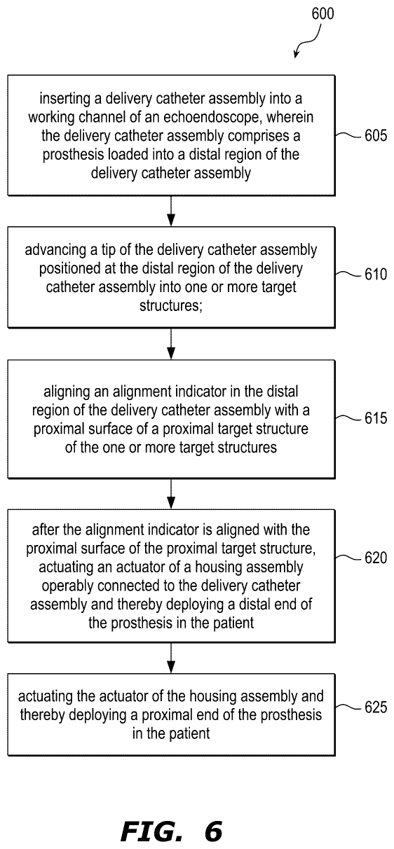

The slide assembly 131 may be configured such that distal movement of the slide assembly 131 moves the delivery catheter assembly 110 in a distal direction and proximal movement of the slide assembly 131 moves the delivery catheter assembly 110 in a proximal direction. For example, in the embodiment illustrated in FIG. 2A, the slide assembly 131 comprises a slide handle 132 configured to slide back and forth over a fixed piston 133. The slide assembly 131 also includes a thumbscrew 134. The lockable slide handle 132 is configured for one-handed operation. The slide handle 132 may include a variety of grip patterns to enhance the ease of grip by the user. A user grasping the slide handle 132 with the right hand (endoscopes are typically configured for left-handed operation) can rotate the thumbscrew 134 with the user's thumb (or thumb and index finger combination) to unlock the slide handle 132. The user can then, with only one hand, slide the slide handle 132 distally over the piston 133. The handle assembly 150 is coupled to the slide handle 132, such that movement of the slide handle 132 also moves the handle assembly 150 and the delivery catheter assembly 110 coupled thereto (discussed below in relation to FIGS. 5A-5H). The slide handle 132 can slide over the piston 133 until it is fully contracted within the slide handle 132. The slide handle 132 can also be proximally retracted and locked into place at any juncture along the piston 133. It should be understood that alternative locking mechanisms to the thumbscrew 134 are encompassed by this disclosure, such as depressible buttons and slidable switches.

A user may adjust the slide assembly 131 under endoscopic ultrasonography (EUS) guidance. An advantage of one-handed operation of the slide assembly 132 is that a user can be watching a video screen and meanwhile easily adjust the position of the delivery catheter assembly 110 with one hand, and still have the other hand (typically the left hand) free for endoscope-related operations.

Referring again to FIG. 2A, in the illustrated embodiment, the housing assembly 130 comprises a rotatable male luer lock adapter 135 configured to mate with a female luer lock adapter (not shown) attached to the working channel of the echoendoscope. The rotatable male luer lock adapter 135 secures engagement of the housing assembly 130 to the working channel of the echoendoscope. The rotatable male luer lock adapter 135 is coupled to the piston 133. More details of the rotatable male luer lock adapter 136 are disclosed in U.S. Pat. No. 11,628,078 filed on Mar. 14, 2018, the disclosure of which is incorporated herein, in its entirety, by this reference.

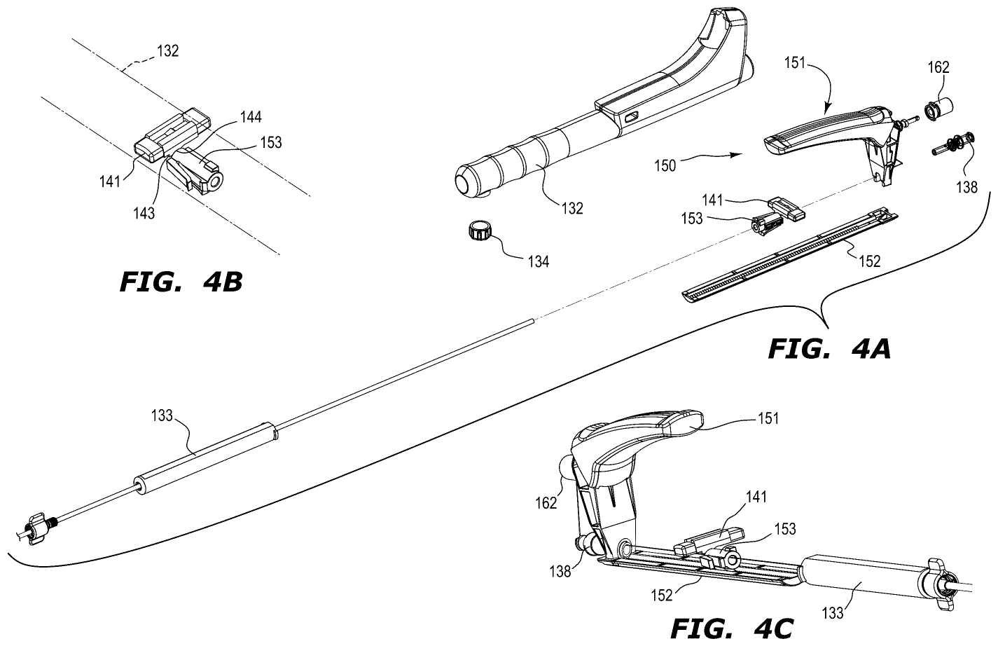

FIG. 2B illustrates an enlarged view of the area 10 of FIG. 1. The catheter assembly 110 includes a tip electrode 170. The tip electrode 170 may be configured for monopolar or bipolar operation. In the illustrated embodiments, the tip electrode 170 is configured for monopolar operation, or in other words, the tip electrode 170 has a single active electrode and the second electrode for completing the circuit is a dispersive electrode (not shown) that is external to the prosthesis delivery device 100. Accordingly, in some embodiments, a conductor (not shown) may run the length of the prosthesis delivery device 100, there being no need for a second conductor as in bipolar operation. More details of the conductor are disclosed in U.S. Pat. No. 11,628,078 filed on Mar. 14, 2018, the disclosure of which is incorporated herein, in its entirety, by this reference. The conductor may be electrically connected to an electrical connection 162 on the handle assembly 150 (shown in FIG. 4A). The electrical connection 162 may be configured for monopolar connection to an electrosurgical power generator (not shown) and may include a 3 mm or 4 mm monopolar post, for example. Likewise, if the tip electrode 170 was configured for bipolar operation, the housing may comprises an electrical connection configured for bipolar connection to the electrosurgical power generator. For example, a second conductor in addition to the conductor may be present. The tip electrode 170 may be configured to quickly cut through tissue walls, such as within about five seconds or less, or about three seconds or less. The tip electrode 170 may be configured to have low friction as the tip electrode 170 is pushed through tissue walls, thereby reducing the force required for passage through the tissue walls. The surface area of the electrode portion of the tip electrode 170 may be minimized so as to focus the current density of the electrode portion.

FIG. 2C illustrates an embodiment of a tip electrode 170. The tip electrode 170 may include a blunt leading edge 172 comprising the electrode portion. For example, the electrode portion of the tip 170 may include a single ring electrode positioned at the blunt leading edge 172. The tip electrode 170 also may include a housing 175 comprising a distal taper 176 to minimize physical resistance as the tip electrode 170 is pushed through tissue. The housing 175 also may include a step 177 that transitions the proximal end of the distal taper 176 to a neck 178. The housing 175 is sized and configured so that an outer sheath 116 (see FIG. 2B) of the delivery catheter assembly 110 can butt up against the step 177 and reside over the neck 178, allowing the outer sheath 116 to be flush with the maximum outer diameter of the housing 175. In some embodiments, the housing 175 further includes the chamfer that tapers the neck 178 to a guidewire sheath (not shown) that extends through the tip electrode 170 and is flush with the leading edge 172. In some embodiments, the guidewire sheath may extend through the tip electrode 170, but may not be flush with the leading edge 172.

The blunt leading edge 172 may be made of an electrically-conductive material, such as a metal. A variety of manufacturing techniques may be used. For example, the blunt leading edge 172 may be machined or metal injection molded The blunt leading edge 172 may be coated with an insulative low-friction coating, such as polytetrafluoroethylene (PTFE) or insulative formulations of ElectroBond or VisiBond, to further focus electrical energy and further focus the current density. For example, a uniform PTFE coating could be applied; however, the coating would not coat/adhere to sharp edges, thereby focusing the electrical current at those sharp edges. Alternatively or additionally, a portion of the surface could be masked to prevent coating adhesion. Instead of or in addition to masking, cutting surfaces could be ground, trimmed, ablated, and/or laser ablated to remove coating material from the cutting surfaces. Additionally, the cutting surfaces could be coated with a conductive low-friction coating, such as conductive formulations of ElectroBond. The housing 175 may be made of a molded insulator, such as a ceramic. Alternatively, the housing 175 may be made of a conductive material and coated with an insulator. In some embodiments, a mask M-1 may be inserted in a proximal end of a neck 178, and the leading edge 172 may be inserted in a mask M-2. The housing 175 may then be coated with an insulator, such as PTFE. The masks M-1 and M-2 may then be removed. In some embodiments, the neck 178 includes a proximal pocket 178a where the conductor may be resistance welded.

More details and other embodiments of tips are disclosed in U.S. Pat. No. 11,628,078 filed on Mar. 14, 2018, the disclosure of which is incorporated herein, in its entirety, by this reference.

In some embodiments, the prosthesis delivery device 100 includes a prosthesis anchor comprising a cylindrical shape and internal lumen configured to be disposed around the guidewire sheath 111 of the prosthesis delivery device 100. The prosthesis anchor may further include a plurality of protuberances that extend radially from the cylinder and are configured to interact with the prosthesis (200 of FIGS. 5C-5D) and secure the prosthesis within a prosthesis pod 117 (shown in FIG. 2B). More details and embodiments of the prosthesis anchor and protuberances are disclosed in U.S. Pat. No. 11,628,078 filed on Mar. 14, 2018, the disclosure of which is incorporated herein, in its entirety, by this reference.

The guidewire sheath 111 may extend to the distal end of the tip electrode 170. In some embodiments, the guidewire sheath 111 extends the entire length of the delivery catheter assembly 110 and the housing assembly 130. In the illustrated embodiments, the housing assembly 130 includes a female luer lock adapter 138 configured for allowing access to the lumen defined by the guidewire sheath 111 (see, e.g., FIGS. 2A and 4A). Referring to FIG. 1, in some embodiments, a mid-sheath circumscribes a proximal region 111a of the guidewire sheath 111 and the prosthesis pod 117 may circumscribe a portion of a distal region 111b of the guidewire sheath 111. The prosthesis pod 117 is configured to receive a prosthesis 200 (in elongated and stretched form). The outer sheath 116 circumscribes the mid-sheath and circumscribes the prosthesis pod 117, according to an embodiment. The outer sheath 116 is translatable over the mid-sheath and the prosthesis pod 117 so as to allow deployment of the prosthesis 200, as is discussed in more detail later. More details and embodiments of the mid-sheath and the prosthesis anchor are disclosed in U.S. Pat. No. 11,628,078 filed on Mar. 14, 2018, the disclosure of which is incorporated herein, in its entirety, by this reference.

As the prosthesis pod 117 is configured to receive the prosthesis 200 in elongated and stretched form, the prosthesis pod 117 may include a region within the delivery catheter assembly 110 that is void of other materials and/or includes space to receive the prosthesis 200 in elongated and stretched form. In some embodiments, the prosthesis pod 117 is positioned in relation to the alignment marker 113a (described in greater detail below). For example, the prosthesis pod 117 may be positioned and configured in the delivery catheter assembly 110 such that when the prosthesis 200 is positioned in the alignment pod 117 before deployment, most (e.g., at least 50%, at least 66%, at least 75%, at least 90%, or at least 95%) of the prosthesis 200 is positioned between the alignment indicator 113a, 113a′ and the distal sheath end 116a.

In some embodiments, the prosthesis 200 includes a distal region configured to form a distal flange 290 (shown in FIG. 5D) when deployed and a proximal region configured to form a proximal flange 280 (shown in FIG. 5D) when deployed. When positioned in the prosthesis pod 117, the proximal region of the prosthesis 200 that forms the proximal flange 280 when deployed may be positioned at least proximate to the alignment indicator 113a, 113a′. More specifically, the proximal region of the prosthesis that forms the distal end of proximal flange 280 when deployed may be positioned in the prosthesis pod 117 to be generally aligned with the indicator 113a, 113a′. In other words, a distal end of the proximal region of the prosthesis (e.g., a transition from the proximal region or flare to the midbody of the prosthesis) that, when deployed, forms the proximal flange 280 may, prior to deployment, be positioned in the prosthesis pod 117 to be generally aligned with the indicator 113a, 113a′.

The guidewire sheath 111, the mid-sheath 112 (and the marker 113), and the outer sheath 116 may be made of a variety of materials. For example, the guidewire sheath 111 may have a three-layer construction with a PTFE-polyimide blend as the inner layer to provide reduced friction with a guidewire, a polyimide middle layer to provide strength without bulk, and a polyether block amide, such as Pebax, outer layer to promote adhesion to the electrode tip 170 and any over-molded features. In some embodiments, the guidewire sheath 111 may not include metallic braiding, so as to minimize capacitive coupling with the conductor; however, in other embodiments, metallic braiding may be present. The mid-sheath may comprise PTFE, given its high dielectric strength, so as to minimize capacitive coupling between the conductor and the guidewire sheath 111 (when the mid-sheath is placed between the two). Other options include a polyether block amide or nylon. The conducting wire may comprise a polyimide coating to minimize coupling. Additionally, the materials of the mid-sheath could have different durometers in different regions to achieve flex-zones in the delivery catheter assembly 110. The mid-sheath may comprises a dual lumen, e.g., a first lumen for the inner sheath and a second lumen for electrical wire. A thin, insulating sleeve or tube may be included within the electrical wire lumen to improve dialectic strength. The outer sheath 116 may have a hydrophilic coating to increase lubricity of the delivery catheter assembly 110. The outer sheath 116 may include PTFE as an inner layer to reduce friction and may include para-aramid fiber braid or axially-oriented fibers to reduce stretching of the delivery assembly 110. The outer sheath 116 may also include materials with different durometers, such as to achieve softer flex-zones. The mid-sheath and the outer sheath 116 may also not include metallic braiding, so as to minimize capacitive coupling.

In some embodiments, the guidewire lumen 111, the mid-sheath, and the outer sheath 116 may be encompassed within the housing assembly 130 by a hypotube or a pair of hypotubes. In some embodiments, the hypotube may be fabricated from stainless steel. In some embodiments, an internal hypotube may be fabricated from stainless steel and an outer hypotube may be fabricated from a non-conductive polymer to help improve the dielectric properties. The outer hypotube may be fabricated from polyether ether ketone (PEEK) or another suitable material. In some embodiments, the stainless steel hypotube may have a polyethylene terephthalate (PET) or PTFE heat shrink to help improve dielectric properties.

The outer sheath 116 may include an alignment indicator 113a spaced from the distal sheath end 116a. For example, in FIG. 2B, the outer sheath 116 includes the marker 113 spaced from the distal sheath end 116a. In this embodiment, the distal end of the marker 113 forms the alignment indicator 113a. Accordingly, in some embodiments, the marker 113 extends proximally on the outer sheath 116 from the alignment indicator 113a. FIGS. 5A-5D show an example of insertion of sheath, partial retraction of the sheath, partial deployment of the prosthesis, and full deployment of the prosthesis utilizing the marker 113 and the alignment indicator 113a.

In other embodiments, the outer sheath 116 may include a marker that extends distally from the alignment indicator. For example, turning to FIG. 2D, the outer sheath 116 includes a marker 113′ spaced from the distal sheath end 116a. In this embodiment, the proximal end of the marker 113′ forms the alignment indicator 113a′, and the marker extend 113′ extends distally from the alignment indicator 113a′. In some embodiments, the marker 113′ extends all the way from the alignment indicator 113a′ to the distal sheath end 116a. In some embodiments, the marker 113a′ extends only partially from the alignment indicator 113a′ to the distal sheath end 116a. FIGS. 5E-5H show an example of insertion of sheath, partial retraction of the sheath, partial deployment of the prosthesis, and full deployment of the prosthesis utilizing the marker 113′ and the alignment indicator 113a′.

When viewed endoscopically, fluoroscopically and/or with the human eye, the outer sheath 116 includes a first color extending proximally from the alignment indicator 113a, 113a′ and a second color different from the first color extending distally from alignment indicator 113a, 113a′. For example, when viewed endoscopically, fluoroscopically and/or with the human eye the marker 113, 113′ may be colored differently than at least the portion of the outer sheath 116 adjacent to marker 113, 113′ (e.g., the rest of the outer sheath 116). In some embodiments, the marker 113, 113′ (including the alignment indicator 113a, 113a′) may be black or green to provide enhanced endoscopic visibility to the user. In some embodiments, the marker 113, 113′ (including the alignment indicator 113a, 113a′) may be black, while at least the portion of the outer sheath adjacent to the marker 113, 113′ may be white. The distal portion of the outer sheath 116 may be transparent other than the marker 113, 113′ that may be opaque (such as via a reflow process) for endoscopic visibility. The transparent distal portion can allow for visibility of the proximal marker 113, 113′ and at least some of the prosthesis pod 117. Metal marker bands may be swaged into or onto the guidewire sheath 111 underneath the marker 113, 113′ (relative to when the outer sheath 116 is fully distally extended, prior to deployment of the prosthesis 200) to provide fluoroscopic visibility.

As shown in FIG. 5B, the alignment indicator 113a may be spaced from the distal sheath end 116a such that when the alignment indicator 113a is aligned with a proximal surface 80a of a proximal target structure 80 (e.g., a stomach wall), the distal sheath end 116a (and the electrode tip 170) are positioned at least proximate to a distal surface 90a of the distal target structure 90 (e.g., a cyst wall). For example, the distal sheath end 116a may be positioned distal to the distal target structure 90. In some embodiments, the alignment indicator 113a may be spaced about 20 mm to about 70 mm from the distal sheath end 116a, such as about 20 mm to about 45 mm, about 45 mm to about 70 mm, about 20 mm to about 40 mm, about 30 mm to about 50 mm, about 40 mm to about 60 mm, or about 50 mm to about 70 mm. The positioning of the alignment indicator 113a′ on the outer sheath 116 may include any of the positioning characteristics of the alignment indicator 113a.

In use, after the distal end 113a has been aligned with the proximal surface 80a of the proximal target structure 80, as the outer sheath 116 is proximally retracted, the alignment indicator 113a (and the marker 113) is also proximally retracted with the outer sheath 116. As illustrated in FIG. 5C, as the outer sheath 116 is retracted about halfway, then the distal half including a distal flange 290 of the prosthesis 200 deploys, illustrated as a stent with flared ends. In some embodiments, a prosthesis anchor 180 may secure the prosthesis 200 within the prosthesis pod 117 during deployment. In some embodiments, the prosthesis anchor may be disposed along the flared end portion (e.g., the distal flange 290) of the prosthesis 200.

As illustrated in FIG. 5D, as the outer sheath 116 is fully retracted, then the proximal half including the proximal flange 280 of the prosthesis 200 deploys. The alignment indicator 113a provides endoscopic and fluoroscopic visibility (when fluoroscopy is used in addition to or instead of EUS) for the proper positioning of the distal region and the tip 170 for deployment of the prosthesis. For example, the alignment indicator 113a (along with the marker 113) may provide endoscopic visibility for the distal region of the delivery catheter assembly 110 as the tip 170 is advanced through the proximal target structure 80 and the distal target structure 90. After the tip 170 has been advanced through the proximal target structure 80 and the distal target structure 90, the endoscopic visibility of the alignment indicator 113a allows the alignment indicator 113a to be aligned with a proximal surface 80a of the proximal target structure 80. In this alignment of the alignment indicator 113a and the proximal surface 80a, the delivery catheter assembly 110 is in the proper position to begin deployment of the prosthesis 200, and a user may lock the prosthesis delivery device 100 to the echoendoscope. More specifically, because the alignment indicator 113a is spaced from the distal sheath end 116a, when alignment indicator 113a is aligned with the proximal surface 80a of the proximal target structure 80, the distal sheath end 116a (and the tip 70) are positioned at least proximate to a distal surface 90a of the distal target structure 90 (e.g., the distal sheath end 116a is positioned distal to the distal target structure 90). This positioning of the alignment indicator 113a aligned with the proximal surface 80a of the proximal target structure 80 indicates the delivery catheter assembly 110 is in the desired positioned to begin deployment of the prosthesis 200, and the delivery catheter assembly 110 may then be locked into this position (e.g., via the thumbscrew 134, described in greater detail below).

With the distal sheath end 116a (and the tip 170) positioned distal to the distal target structure 90, actuating an actuator or lever 151 on the handle assembly 150 that moves the outer sheath 116 distally relative to the tip 170 deploys a distal prosthesis end including the distal flange 290 of the prosthesis 200 in the patient at least proximate (e.g. adjacent) to a distal surface 90a of the distal target structure 90. The actuator may be further actuated to further move the outer sheath 116 relative to the stationary actuator tip 170, thereby deploying a proximal prosthesis end including the proximal flange 280 of the prosthesis 200 at least proximate (e.g., adjacent) to the proximal surface 80a of the proximal target structure 80.

Referring back to FIGS. 2A, the housing assembly 130 may include a safety tab 141 (e.g., button). In some embodiments, the housing assembly 130 includes only a single safety tab 141 For example, the single safety tab 141 may be the only safety tab present on the housing assembly and/or handle assembly 150, and additional safety tabs are absent from the housing assembly 130 and/or the handle assembly 150. The safety tab 141 prevents deployment of the distal portion of the prosthesis 200 by the handle assembly 150. In some embodiments, the single safety tab 141 is the only safety tab operably coupled to and/or configured to lock and/or unlock the actuator or lever 151 of the handle assembly 150. Exemplary operation of the handle assembly 150 is now described.

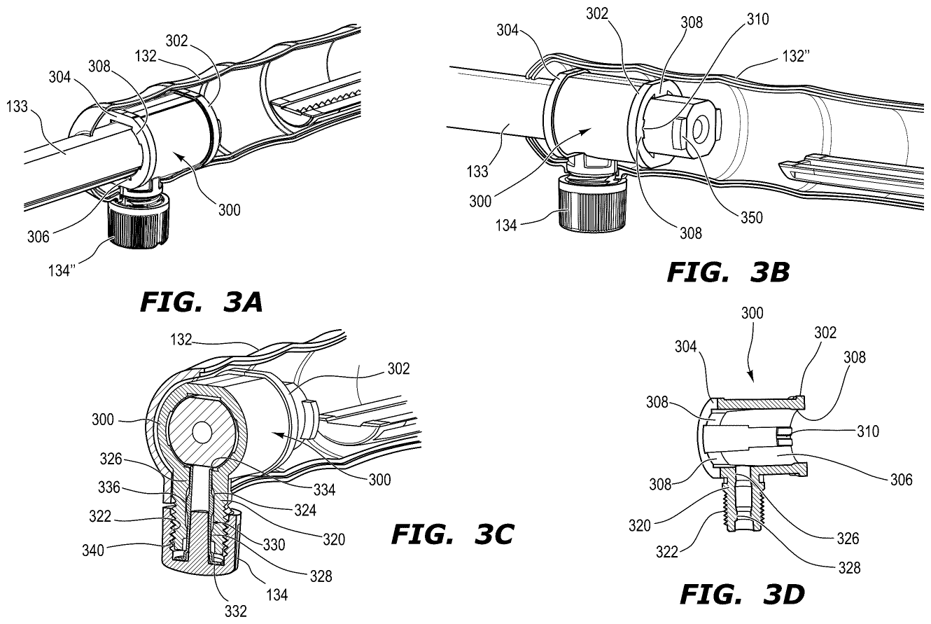

FIG. 3A illustrates a breakaway view of the thumbscrew 134 from a distal view and FIG. 3B illustrates a breakaway view of the thumbscrew 134 from a proximal view. A bushing 300 disposed within the slide handle 132 may be disposed around the piston 133. The bushing 300 and the piston 133 may be coaxial with the slide handle 132 and may be disposed along the longitudinal axis of the slide handle 132. The bushing 300 may be configured to stabilize the piston 133 and to keep the piston 133 in coaxial alignment with the slide handle 132. The bushing 300 extends between a proximal end 302 and a distal end 304 and defines an internal lumen 306 configured to be disposed around the piston 133. The bushing 300 may further include a plurality of pads 308 that project radially inward from the internal lumen 306 and the pads 308 may engage with the piston 133 to create a tight fit and/or to maintain the coaxial alignment of the piston 133 and the slide handle 132. The pads 308 are not configured to prevent longitudinal movement of the piston 133 but to create a minimal clearance fit to prevent or minimize the piston 133 from moving out of alignment with the slide handle 132. Thus, the pads 308 are configured to stabilize the piston 133 in coaxial alignment including during longitudinal displacement of the piston 133. The pads 308 on the distal end 304 are shown in the view of FIG. 3A and the pads 308 on the proximal end are shown in the view of FIG. 3B. In some embodiments, there may be four pads 308 on the proximal end 302 and four pads 308 on the distal end. However, there may be more or less than four pads 308 on each end. In some embodiments, there may be an unequal numbers of pads 308 on the proximal end 302 compared to the distal end 304.

In some embodiments the pads 308 are equally spaced around the internal circumference of the internal lumen 306. In some embodiments, the pads 308 on the proximal end 302 and the pads 308 on the distal end 304 may be misaligned with each other. In some embodiments, the pads 308 on the proximal end 302 and the pads 308 on the distal end 304 may be aligned with each other.

In some of the embodiments, such as illustrated in FIG. 3B, some of the pads 308 may include a groove 310 that is centrally disposed on the pad 308. The groove 310 is configured to enable a low clearance fit between the pad 308 and the piston 133 around the majority of the circumference of the piston 133 while also allowing increased tolerance along parting lines or other features of the piston 133, for example to allow greater tolerance for flashing on the piston 133 or other manufacturing artifacts or features.

In some embodiments, such as illustrated in FIG. 3B, the piston 133 may further include a plurality of projections 350 that project radially outward from a proximal end of the piston 133. The projections 350 are configured to interact with the bushing 300 and prevent the piston 133 from moving too far in a distal direction relative to the slide handle 132.

FIG. 3C illustrates a breakaway cross-sectional view of the thumbscrew 134 and the bushing 300. The bushing 300 may further include a projection 320 that extends from an outer surface of the bushing 300. The projection may include threads 322 configured to interacts with corresponding threads 340 on the thumbscrew 134. The projection 320 may further include an internal lumen 324, such as a lumen centrally disposed within the projection 320. The internal lumen 324 of the projection may be in communication with the lumen 306 of the bushing 300. The internal lumen 324 may include multiple narrow portions. For example, internal lumen 324 may include a first narrow portion 326 that is proximal of the internal lumen 306 of the bushing 300 and a second narrow portion 328 that is distal of the internal lumen 306 of the bushing 300.

The thumbscrew 134 may further include a locking feature 330. The locking feature may include a first end 332 and a second end 334, the first end 332 may be coupled to an internal surface of the thumbscrew 134. The second end 334 is engagable with the piston 133. In use, a user may turn the thumbscrew to advance the second end 334 of the locking feature into engagement with the piston 133. When the locking feature 330 is thus engaged with the piston 133, the locking feature 330 may prevent or minimize movement of the piston 133 relative to the slide handle 132. The user may turn the thumbscrew in an opposite direction to retract the locking feature 330 and disengage the locking feature 330 from the piston 133 to facilitate longitudinal displacement of the piston 133 relative to the slide handle 132.

The locking feature 330 may further include a bulbous or bulging feature 336 that bulges or projects radially outward from the locking feature 330. The bulging feature 336 may be disposed between the first narrow portion 326 and the second narrow portion 328 of the internal lumen 324 of the projection 320. The bulging feature 336 may thus tend to maintain the thumbscrew 134 coupled to the bushing 300 and to the slide handle 132 and prevent accidental uncoupling of the thumbscrew 134 from the bushing 300 and the slide handle 132. In other words, the thumbscrew 134 may be rotated such that the thumbscrew 134 allows for longitudinal displacement of the piston 133 and the bulging feature 336 prevents the thumbscrew 134 from falling off or otherwise decoupling from the entire assembly. The thumbscrew 134 may be uncoupled from the bushing and the slide handle 132 if a sufficient force is applied to displace the bulging feature 336 past the second narrow portion 328 of the internal lumen 324 of the projection 320.

FIG. 3D illustrates a perspective cross-sectional view of the bushing 300. The pads 308 are shown extending in the longitudinal direction of the bushing 300. The length of the pads in the longitudinal direction may vary. In some embodiments, the pads 308 interface with the piston 133, but the internal lumen 306 does not directly contact or interface with the fixed piston. Groove 310 of some of the pads 308 extend longitudinally as well.

FIG. 4A illustrates an exploded view of the housing assembly 130. The handle assembly 150 includes an actuator or lever 151, a track 152, and a sheath adapter 153, according to an embodiment. The actuator 151 may be configured to pivot about a pivot point when actuated. A spring 145 may engage with the actuator 151 when the actuator 151 is depressed. The spring 145 may return the actuator 151 to its unpivoted position when the actuator 151 is no longer depressed by a user. As the actuator 151 is depressed and pivots, it engages with the track 152 and moves the track 152 proximally (unless the safety button 141′ is not pressed, as is discussed below). The track 152 may be engaged with the sheath adapter 153. As the track 152 proximally retracts, the sheath adapter 153 may thus follow with the track 152. The sheath adapter 153 may be fixedly coupled to the outer sheath 116. Proximal movement of the sheath adapter 153 therefore results in proximal movement of the outer sheath 116. Partial retraction of the sheath adapter 153 results in deployment of the distal half of the prosthesis. Complete retraction of the sheath adapter 153 results in full deployment of the prosthesis.

The safety button 141 illustrated in FIGS. 4B and 4C is a non-detachable push buttons configured to interact with the sheath adapter 153 when the safety button 141 is not pressed. The safety button 141 includes a protrusion 143 configured to interact with the sheath adapter 153, according to an embodiment. When the safety button 141 is not pressed, the sheath adapter 153 is unable to move proximally when the actuator 151 is depressed. When the safety button 141 is pressed, the safety button 141 slides in a direction orthogonal to the longitudinal direction of the prosthesis delivery device 100 and the protrusion 143 is configured to no longer engage with the sheath adapter 153. With the safety button 141 engaged, the sheath adapter 153′ is able to move proximally when the actuator 151 is depressed.

One of the benefits of the illustrated embodiments of the prosthesis delivery device 100 is that the handle assembly 150 may be completely operated with one hand, including depression or activation of the safety tab 141.

In the illustrated embodiment, it is not possible to depress the actuator 151 when the safety tab 141 is in place. In other embodiments, the handle assembly 150 may be configured such that depressing the actuator 151 is possible, but has no effect unless the safety tab 141 is depressed or activated. Additionally, the track 152 may be configured so that each full ratchet of the track 152 by the actuator 151 retracts the outer sheath 116 about 1 cm to about 2 cm. In some embodiments, the track 152 may be modified to provide much smaller increments of movement over an entire depression of the actuator 151 or to provide small movements of the outer sheath 116 as the actuator 151 is partially depressed. For example, a full ratchet may retract the outer sheath 116 about 1 cm, but partial ratchet may move the outer sheath 116 in 2 mm increments until it reaches a full ratchet and movement of 1 cm. This may enable the user to have greater precision when deploying the prosthesis. It should be understood that the illustrated embodiment is just one approach to proximally retracting the outer sheath 116. One of ordinary skill in the art, with the benefit of this disclosure, would understand that a number of approaches may be used for retracting the outer sheath 116.

It should be understood that the prosthesis delivery device 100 will normally be supplied as a kit with a prosthesis, such as the prosthesis 200, loaded into the prosthesis pod 117; however, that may not always be the case.

FIG. 6 is a flow diagram of a method 600 of deploying a prosthesis in a patient, according to an embodiment. The method 600 include use of any embodiments of the prosthesis delivery device 100 and/or delivery catheter assembly 110 described herein. In an embodiment, the method 600 may an act 605 of inserting a delivery catheter assembly 110 into a working channel of an echoendoscope. The delivery catheter assembly 110 comprises a prosthesis 200 loaded into a distal region of the delivery catheter assembly 110. The method 600 may include an act 610 of advancing a tip 170 of the delivery catheter assembly 110 positioned at the distal region of the delivery catheter assembly 110 into one or more target structures (shown in FIGS. 5A and 5E). The method 600 may include an act 615 of, after advancing the tip 170 into the one or more target structures, aligning an alignment indicator 113a, 113a′ in the distal region of the delivery catheter assembly 110 with a proximal surface 80a of a proximal target structure 80 of the one or more target structures (shown in FIGS. 5B and 5F). The method 600 may include an act 620 of, after the alignment indicator 113a, 113a′ is aligned with the proximal surface 80a of the proximal target structure 80, actuating an actuator 151 of a housing assembly 130 operably connected to the delivery catheter assembly and thereby deploying a distal end of the prosthesis 200 in the patient (shown in FIGS. 5C and 5G). The method 600 may include an act 625 of actuating the actuator 151 of the housing assembly 130 and thereby deploying a proximal end of the prosthesis 200 in the patient (shown in FIGS. 5D and 5H).

In some embodiments of the method 600, the method 600 further comprises an act of locking the housing assembly 130 to the echoendoscope after the act 605 and prior to the act 610. For example, the method 600 may include an act of rotating (e.g., rotating clockwise) the male luer lock adapter 135 on the housing assembly 130 and thereby securing the housing assembly 130 to the echoendoscope prior to advancing the tip 170 of the delivery catheter assembly 110 into the one or more target structures.

In some embodiments, the method 600 may include an act of connecting the prosthesis delivery device 100 to a power source. For example, the method 600 may include an act of connecting the prosthesis delivery device 100 to a radiofrequency generator via a cable, and ensuring the generator has a proper setting selected.

In some embodiments, prior to the act 610, the method 600 may include an act of positioning the EUS scope at least proximate to the proximal structure 80 (e.g., the stomach wall) in the desired location for electrosurgical cutting into the proximal structure 80. Accordingly, the method 600 may include an act of confirming visually via fluoroscopic or ultrasound that the echoendoscope is positioned at a selected position proximate to the proximal surface 80a of the proximal target structure 80 prior to advancing the tip 170 of the delivery catheter assembly 110 into the one or more target structures.

In some embodiments, prior to the act 610, the method 600 may include an act of loosening a lock on the housing assembly 130 to allow movement of the tip 170 relative to the echoendoscope. For example, the method 600 may include an act of rotating the thumbscrew 134 to loosen the thumbscrew and unlock the lockable slide handle 132 prior to advancing the tip 170 of the delivery catheter assembly 110 into the one or more target structures.

In some embodiments, method 600 includes an act of energizing the tip 170 prior the act 610 of advancing the tip 170 into one or more target structures. For example, the method 600 may include an act of energizing a single ring electrode on the tip 170 of the delivery catheter assembly 110 prior to advancing the tip 170 into the one or more target structures. The method also may include an act of de-energizing the single ring electrode on the tip 170 after penetrating the one or more target structures. The act 610 may includes advancing the tip 170 through the proximal target structure 80 and a distal target structure 90.

In some embodiments, after the tip 170 has been advanced through the one or more target structures, the method 600 may include acts of tighten the thumbscrew 134, powering off the generator, and/or unplugging the cable from the device 100. The method 600 may further comprise loosening the thumbscrew 134 to allow the tip 170 and distal region of the delivery catheter assembly 110 to move relative to the echoendoscope.

The method 600 may include an act of positioning the tip 170 of the delivery catheter assembly in a selected position relative to the one or more target structures. For example, as noted above, the method 600 includes the act 615 of aligning the alignment indicator 113a, 113a′ in the distal region of the delivery catheter assembly 110 with a proximal surface 80a of a proximal target structure 80 of the one or more target structures. More particularly, the act 615 may include pulling and/or pushing the housing assembly 130 until the alignment indicator 113a, 113a′ is aligned with the proximal surface 80a of the proximal target structure 80. The delivery catheter assembly 110 may be movable relative to the echoendoscope as the thumbscrew 134 is loosened. In some embodiments, the act 615 includes pulling and/or pushing the delivery catheter assembly proximally until the alignment indicator 113a, 113a′ on the outer sheath 116 of the delivery catheter assembly 110 is aligned with the proximal surface 80a of the proximal target structure 80 of the one or more target structures. In embodiments including the marker 113 extending proximally from the alignment indicator 113a, proper alignment of the alignment indicator 113a for deployment of the prosthesis 200 requires substantially all of the marker 113 to be visible adjacent to the proximal target 80, but not disposed in the hole formed in the proximal target 80 by the tip 170 (shown in FIG. 5B).

In embodiments including the marker 113′ extending distally from the alignment indicator 113a′, proper alignment of the alignment indicator 113a′ for deployment of the prosthesis requires only the proximal end of the marker 113′ to be visible adjacent to the proximal target 80 (shown in FIG. 5F), but not disposed in the hole formed in the proximal target 80 by the tip 170.

In some embodiments, after the alignment indicator 113a, 113a′ is aligned with the proximal surface 80a of the proximal target structure 80 and prior to actuating the actuator 151, the method 600 includes an act of preventing movement of the tip 170 distal or proximal relative to the echoendoscope. For example, the method include an act of rotating (e.g. tightening) the thumbscrew 134 on the housing assembly 130 and thereby preventing movement of the tip 170 distally or proximally relative to the echoendoscope.

In some embodiments, after the thumbscrew 134 is rotated (e.g. tightened) to prevent movement of the tip 170 relative to the echoendoscope and prior to actuating the actuator 151, the method 600 includes an act of pressing the safety button 141 to unlock the actuator 151. The safety button 141 may be the only safety button present on the housing assembly 130. For example, the method 600 may include an act of pressing the safety button 141 to unlock the actuator 151 for prosthesis 200 deployment. In some embodiments, the safety button 141 is absent. In some embodiments, the method 600 includes an act of engaging the safety button 141 on the housing assembly 130 or ensuring the safety button 141 is engaged prior to advancing the tip 170 of the delivery catheter assembly 110 into the one or more target structures.

As noted above, the method 600 includes an act 620 of, after the alignment indicator 113a, 113a′ is aligned with the proximal surface 80a of the proximal target structure 80, actuating the actuator 151 and thereby deploying a distal end of the prosthesis 200 in the patient, and also an act 625 of actuating the actuator 151 and thereby deploying a proximal end of the prosthesis 200 in the patient. More specifically, the act 620 may include actuating the actuator 151, thereby retracting the outer sheath 116 a predetermined distance from the tip 170 and deploying the distal end of the prosthesis 200 in the patient. The act 625 may include actuating the actuator 151, thereby retracting the outer sheath 116 an additional predetermined distance (which may be the same as the predetermined distance of the act 620) from the tip 170 and deploying the proximal end of the prosthesis 200 in the patient. More specifically, the act 620 may include actuating the actuator 151 and thereby deploying the distal end of the prosthesis 200 such that the prosthesis 200 forms a distal flange 290 at least proximate to a distal surface 90a of the distal target structure 90 and the act 625 may include actuating the actuator 151 and thereby deploying the proximal end of the prosthesis 200 such that the prosthesis forms a proximal flange 280 at least proximate to a proximal surface 80a of the proximal target structure 80. For example, the method 600 may include squeezing the lever of the actuator 151 repeatedly to retract the outer sheath 116 relative to the tip 170 and deploy the stent while maintaining position of the catheter.

In some embodiments, the method 600 includes an act of, after actuating the actuator 151 and thereby deploying the proximal end of the prosthesis 200 in the patient, rotating unlocking the housing assembly from the echoendoscope and pulling the delivery catheter assembly 110 from the working channel of the echoendoscope. For example, once prosthesis is fully deployed, the method 600 may include rotating the male luer lock 135 counterclockwise to release the housing assembly 130 from the echoendoscope and pull the delivery catheter assembly 110 out of the working channel in a slow motion.

Acts of the method 600 are for illustrative purposes. For example, the acts of the method 600 may be performed in different orders, split into multiple acts, modified, supplemented, or combined. Any of the acts may include using any of the catheter deliver devices disclosed herein.

The prosthesis delivery devices and methods of delivery a prosthesis disclosed herein may be used for a variety of procedures. For example, any time it is desirable to deploy a prosthesis in two stages, but with only one-hand, then it may be beneficial to use the prosthesis delivery devices and methods disclosed herein. For example, the prosthesis delivery devices and methods disclosed herein may be used for draining one lumen of a patient into another lumen of a patient, such as, for example, transgastric or transduodenal drainage of a pancreatic pseudocyst, of a biliary tract, of a gallbladder. An access port may be created with the tip electrode between a first lumen of the patient and a second lumen of the patient. The first lumen may be the gastrointestinal tract (for example, the esophagus, stomach, pylorus, or bowel) of the patient. The second lumen may be the gallbladder, a pancreatic cyst, a biliary tract, or some other lumen that needs drainage. In some embodiments, the prosthesis delivery devices and methods disclosed herein may be used in a bariatric procedure (e.g., stomach to stomach).

In an example, draining a target structure of a patient may include introducing an echoendoscope into the gastrointestinal tract of the patient with the terminal end in the vicinity of the target structure. A guidewire may be inserted through the working channel of the echoendoscope into the target structure. The delivery catheter assembly 110 (with a prosthesis loaded into the prosthesis pod 117) may then be slid over the guidewire and into the working channel of the echoendoscope. Alternatively, a guidewire may not be present, and instead the location of the terminal end of the echoendoscope is sufficiently precisely positioned near the target structure so as to guide placement of the delivery catheter assembly 110. The housing assembly 130 may be secured to the echoendoscope. The tip electrode 170 may extend just beyond the terminal end of the echoendoscope. When using the housing assembly 130, with one-hand, while watching the endoscope video screen, the user may unlock the handle 132, energize the tip electrode 170 (such as by depressing a foot pedal connected to the electrosurgical power generator), slide the handle 132 distally until the target structure has been penetrated and an access port created. In some embodiments, the method may include a step of tenting the tissue prior to energizing the electrode tip 170. The electrode tip 170 may then be deenergized (such as by releasing the foot pedal). The handle 132 may then be locked in place with the same hand. In some embodiments, with the same hand as before, the safety tab 141 may be removed from the housing assembly 130. With the same hand the actuator 151 may then be depressed and thereby deploy the distal end of the prosthesis in the target structure of the patient. With the same hand, the housing assembly 130 may be retracted proximally (and thereby the entire delivery catheter assembly 110 and the partially deployed prosthesis) to confirm visually either endoscopically, fluoroscopically, or via ultrasound that the distal end of the prosthesis is secured against the tissue wall inside the target structure. With the same hand the actuator 151 may again be depressed and thereby deploy the proximal end of the prosthesis in the gastrointestinal tract of the patient. With the same hand the handle 132 may be unlocked and proximally retracted to withdraw the electrode tip 170 from the target structure. The prosthesis, such as the prosthesis 200, can now allow drainage of the target structure.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, only a portion of a method described herein may be a separate method. Stated otherwise, some methods may include only a portion of the steps described in a more detailed method.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of this disclosure.

1. A method of deploying a prosthesis in a patient, the method comprising:
inserting a delivery catheter assembly into a working channel of an echoendoscope, wherein the delivery catheter assembly comprises a prosthesis loaded into a distal region of the delivery catheter assembly;
advancing a tip of the delivery catheter assembly positioned at the distal region of the delivery catheter assembly into one or more target structures;
after advancing the tip into the one or more target structures, aligning an alignment indicator in the distal region of the delivery catheter assembly with a proximal surface of a proximal target structure of the one or more target structures;
after the alignment indicator is aligned with the proximal surface of the proximal target structure, actuating an actuator of a housing assembly operably connected to the delivery catheter assembly and thereby deploying a distal end of the prosthesis in the patient; and
actuating the actuator of the housing assembly and thereby deploying a proximal end of the prosthesis in the patient.

2. The method of claim 1, further comprising, after the alignment indicator is aligned with the proximal surface of the proximal target structure and prior to actuating the actuator, rotating a first lock on the housing assembly and thereby preventing movement of the tip distally or proximally relative to the echoendoscope.

3. The method of claim 2, further comprising:
engaging a safety button on the housing assembly prior to advancing the tip of the delivery catheter assembly into the one or more target structures, the safety button being the only safety button present on the housing assembly; and
after the first lock is rotated to prevent movement of the tip relative to the echoendoscope and prior to actuating the actuator, pressing the safety button to unlock the actuator.

4. The method of claim 2, further comprising:
confirming visually via fluoroscopic or ultrasound that the echoendoscope is positioned at a selected position proximate to the proximal surface of the proximal target structure prior to advancing the tip of the delivery catheter assembly into the one or more target structures; and
after actuating the actuator of the housing assembly and thereby deploying the proximal end of the prosthesis in the patient, rotating a second lock to release the housing assembly from the echoendoscope and pulling the delivery catheter assembly from the working channel of the echoendoscope.

5. The method of claim 4, further comprising:
rotating the second lock on the housing assembly and thereby securing the housing assembly to the echoendoscope prior to advancing the tip of the delivery catheter assembly into the one or more target structures; and
rotating the first lock to loosen the first lock prior to advancing the tip of the delivery catheter assembly into the one or more target structures.

6. The method of claim 1, wherein:
aligning an alignment indicator in the distal region of the delivery catheter assembly with a proximal surface of a proximal target structure of the one or more target structures includes aligning the alignment indicator on an outer sheath of the delivery catheter assembly with the proximal surface of the proximal target structure of the one or more target structures;
actuating an actuator of a housing assembly operably connected to the delivery catheter assembly and thereby deploying a distal end of the prosthesis in the patient includes actuating the actuator, thereby retracting the outer sheath a predetermined distance from the tip and deploying the distal end of the prosthesis in the patient; and
actuating the actuator of the housing assembly and thereby deploying a proximal end of the prosthesis in the patient includes actuating the actuator of the housing assembly, thereby retracting the outer sheath an additional predetermined distance from the tip and deploying the proximal end of the prosthesis in the patient.

7. The method of claim 1, wherein the actuator includes a handle assembly on the housing assembly.

8. The method of claim 1, further comprising energizing an electrode ring on the tip of the delivery catheter assembly prior to advancing the tip into the one or more target structures and then de-energizing the electrode ring after penetrating the one or more target structures.

9. The method of claim 1, wherein:
advancing a tip of the delivery catheter assembly positioned at the distal region of the delivery catheter assembly into one or more target structures includes advancing the tip through the proximal target structure and a distal target structure;
actuating an actuator of a housing assembly operably connected to the delivery catheter assembly and thereby deploying a distal end of the prosthesis in the patient includes actuating the actuator and thereby deploying the distal end of the prosthesis at least proximate to a distal surface of the distal target structure in the patient; and
actuating the actuator of the housing assembly and thereby deploying a proximal end of the prosthesis in the patient includes actuating the actuator of the housing assembly and thereby deploying the proximal end of the prosthesis at least proximate to the proximal surface of the proximal target structure.

10. The method of claim 9, wherein the distal target structure includes a cyst wall and the proximal target structure includes a stomach wall position at least proximate to the cyst wall.

11. A method of deploying a prosthesis in a patient, the method comprising:
inserting a delivery catheter assembly into a working channel of an echoendoscope, wherein the delivery catheter assembly comprises a prosthesis loaded into a distal region of the delivery catheter assembly;
advancing a tip of the delivery catheter assembly positioned at the distal region of the delivery catheter assembly into one or more target structures;
positioning the tip of the delivery catheter assembly in a selected position relative to the one or more target structures;
after the tip is positioned in the selected position, rotating a first lock on the housing assembly and thereby preventing movement of the tip distally or proximally relative to the echoendoscope;
after rotating the first lock and thereby preventing movement of the tip, actuating an actuator of a housing assembly operably connected to the delivery catheter assembly and thereby deploying a distal end of the prosthesis in the patient; and
actuating the actuator of the housing assembly and thereby deploying a proximal end of the prosthesis in the patient.

12. The method of claim 11, wherein positioning the tip of the delivery catheter assembly in a selected position relative to the one or more target structures includes aligning an alignment indicator in the distal region of the delivery catheter assembly with a proximal surface of a proximal target structure of the one or more target structures.

13. The method of claim 12, wherein:
aligning an alignment indicator in the distal region of the delivery catheter assembly with a proximal surface of a proximal target structure of the one or more target structures includes aligning the alignment indicator on an outer sheath of the delivery catheter assembly with the proximal surface of the proximal target structure of the one or more target structures;
actuating an actuator of a housing assembly operably connected to the delivery catheter assembly and thereby deploying a distal end of the prosthesis in the patient includes actuating the actuator, thereby retracting the outer sheath a predetermined distance from the tip and deploying the distal end of the prosthesis in the patient; and
actuating the actuator of the housing assembly and thereby deploying a proximal end of the prosthesis in the patient includes actuating the actuator of the housing assembly, thereby retracting the outer sheath an additional predetermined distance from the tip and deploying the proximal end of the prosthesis in the patient.

14. (canceled)

15. The method of claim 11, further comprising:
confirming visually via fluoroscopic or ultrasound that the echoendoscope is positioned at a selected position proximate to the proximal surface of the proximal target structure prior to advancing the tip of the delivery catheter assembly into the one or more target structures; and
after actuating the actuator of the housing assembly and thereby deploying the proximal end of the prosthesis in the patient, rotating a second lock to release the housing assembly from the echoendoscope and pulling the delivery catheter assembly from the working channel of the echoendoscope.

16. The method of claim 15, further comprising:
rotating the second lock on the housing assembly and thereby securing the housing assembly to the echoendoscope prior to advancing the tip of the delivery catheter assembly into the one or more target structures; and
rotating the first lock to loosen the first lock prior to advancing the tip of the delivery catheter assembly into the one or more target structures.

17-20. (canceled)

21. A method of deploying a prosthesis in a patient, the method comprising:
inserting a delivery catheter assembly into a working channel of an echoendoscope, wherein the delivery catheter assembly comprises a prosthesis loaded into a distal region of the delivery catheter assembly;
advancing a tip of the delivery catheter assembly positioned at the distal region of the delivery catheter assembly into one or more target structures;
positioning the tip of the delivery catheter assembly in a selected position relative to the one or more target structures;
after positioning the tip of the delivery catheter assembly in the selected position, pressing a safety button on the housing assembly to unlock the actuator, the safety button being the only safety button present on the housing assembly that is operably coupled to the actuator;
after positioning the tip of the delivery catheter assembly in the selected position and after unlocking the actuator, actuating an actuator of a housing assembly operably connected to the delivery catheter assembly and thereby deploying a distal end of the prosthesis in the patient; and
actuating the actuator of the housing assembly and thereby deploying a proximal end of the prosthesis in the patient.

22. The method of claim 21, wherein positioning the tip of the delivery catheter assembly in a selected position relative to the one or more target structures includes aligning an alignment indicator in the distal region of the delivery catheter assembly with a proximal surface of a proximal target structure of the one or more target structures.

23. (canceled)

24. The method of any of claim 21, further comprising,
after positioning the tip of the delivery catheter assembly in the selected position and prior to actuating the actuator, rotating a first lock on the housing assembly and thereby preventing movement of the tip distally or proximally relative to the echoendoscope.

25. The method of claim 24, further comprising:
confirming visually via fluoroscopic or ultrasound that the echoendoscope is positioned at a selected position proximate to the proximal surface of the proximal target structure prior to advancing the tip of the delivery catheter assembly into the one or more target structures; and
after actuating the actuator of the housing assembly and thereby deploying the proximal end of the prosthesis in the patient, rotating a second lock to release the housing assembly from the echoendoscope and pulling the delivery catheter assembly from the working channel of the echoendoscope.

26. The method of claim 25, further comprising:
engaging the safety button on the housing assembly prior to advancing the tip of the delivery catheter assembly into the one or more target structures; and
rotating the second lock on the housing assembly and thereby securing the housing assembly to the echoendoscope prior to advancing the tip of the delivery catheter assembly into the one or more target structures; and
rotating the first lock to loosen the first lock prior to advancing the tip of the delivery catheter assembly into the one or more target structures.

27-30. (canceled)
